(19) United States
(12) Patent Application Publication
(10) Pub. No.: US 2025/0179002 A1
(43) Pub. Date: Jun. 5, 2025

(54) PROCESS FOR THE PRODUCTION OF FATTY ACIDS AND ISOMERIZED ESTERS

(71) Applicant: NUOL Green Chemistry LLC, Little Falls, MN (US)

(72) Inventor: Setsuo Sato, Piracicaba (BR)

(21) Appl. No.: 18/596,120

(22) Filed: Mar. 5, 2024

(30) Foreign Application Priority Data
Nov. 30, 2023 (BR) 10 2023 025232 0

(51) Int. Cl. C07C 67/347; B01J 29/70; C07C 67/54; C08G 69/26

(52) U.S. Cl. CPC C07C 67/347; B01J 29/7007; C07C 67/54; C08G 69/26

(57) ABSTRACT

The development of isomerized processes and products using triglycerides rich in linear oleic and linoleic chains as raw materials is provided. Isomerization physically and chemically modifies fatty acids and methyl esters, creating opportunities for new applications that are not possible with linear chain materials. Chains with methyl radicals provide a lower melting point, greater adhesion (see properties between polyethylene and polypropylene polymers), excellent emollience and spreadability on physical surfaces such as skin, leaves, wood, hair. Monoesters, diesters, triesters, tetraesters are produced with a range of possible applications previously not possible with linear fatty materials.

The invention is based on the development of isomerized processes and products using triglycerides rich in linear oleic and linoleic chains as raw materials. Isomerization physically and chemically modifies fatty acids and methyl esters, creating opportunities for new applications that are not possible with linear chain materials. Chains with methyl radicals provide a lower melting point, greater adhesion (see properties between polyethylene and polypropylene polymers), excellent emollience and spreadability on physical surfaces such as skin, leaves, wood and hair.

### BACKGROUND OF THE INVENTION

Isomerized fatty acids do not exist in vegetable oils and they must be synthetically modified, as shown in the figure below:

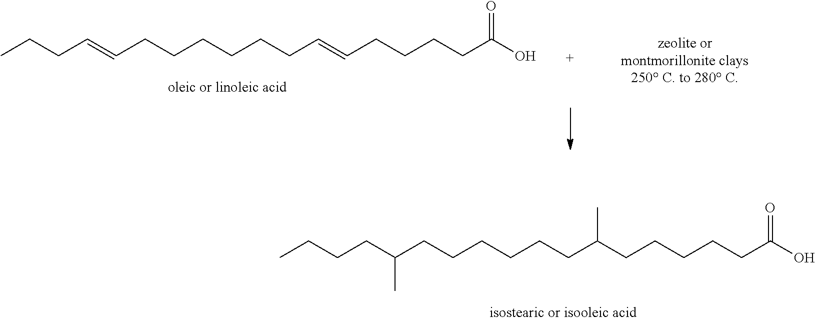 [SMILES: CCC/C=C/CCCCCC/C=C/CCCCC(=O)O ; CCCCC(C)CCCCCCC(C)CCCCCC(=O)O]

The production of isomerized acids received a major boost when the production of dimeric and trimeric acids began, which are used in the production of polyamides for the adhesives and printing inks segment. The isomerized acids of this process are co-products whose amount formed is approximately 50% by weight

U.S. Pat. No. 6,281,373 B1 describes the production of dimeric acids from soybean oil and vegetable oil distillate, known in the market as VOD. It describes the production of esters, dimerization, separation of isomerized fatty acids and the production of polyamides.

Patent KR 100356436B1 mentions the production of trimeric acids from distilled fatty acids using montmorrilonite clay and aluminum chloride as catalysts, resulting in a product with 52% isomerized fatty acids, 45% trimeric acids and 3% dimeric acids.

U.S. Pat. No. 2,731,481A cites the production of dimeric acids using oleic acid as raw material and organic peroxides with the aim of obtaining high levels of dimers and low levels of trimers. Reaction temperatures are between 100° C. and 130° C.

### DETAILED DESCRIPTION OF THE INVENTION

The Figure below shows the formation of isomerized fatty acids and lactones from oleic acid using a commercial Beta zeolite as a catalyst (calcined at 450° C. instead of 550° C. to reduce its porosity).

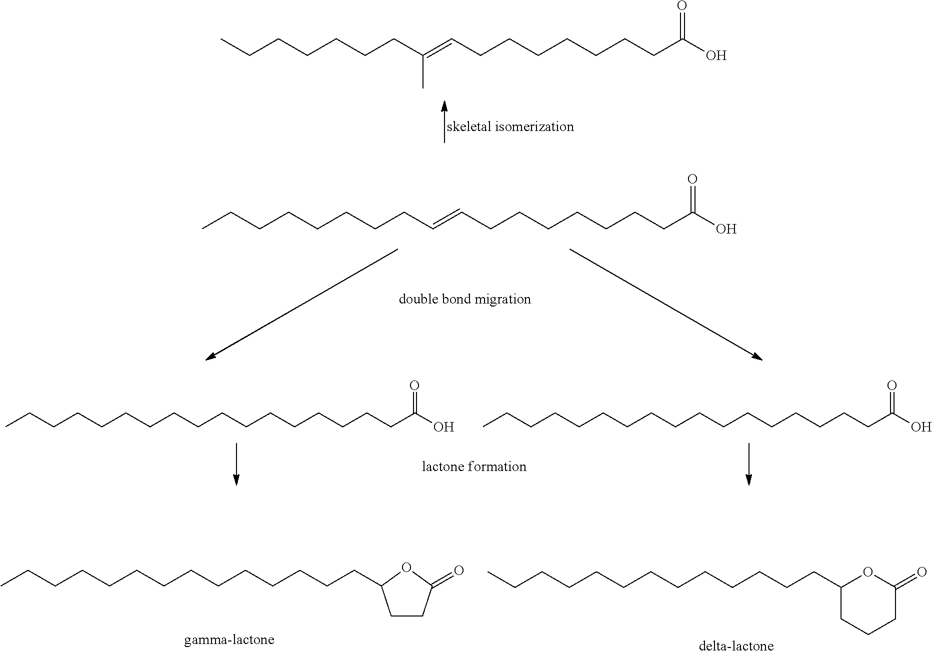

### Examples

### Isomerization of Talloil Fatty Acids:

a. In a 2-liter autoclave, 1000 grams of talloil fatty acid (TOFA) were charged;
b. Added 70 grams of montmorillonite B clay and 5 grams of lithium hydroxide;
C. Pressurized with 5 kg of nitrogen;
d. Reactor temperature was gradually increased until reaching 260° C.;
e. During the heating process the reactor pressure was relieved to maintain the pressure at 5 bar;
f. Product was reacted for 4 hours at 260° C.;
g. After this period it was cooled to 90° C. and filtered;
h. Product composition 49.2% monomers, 44.7% dimers, 6.1% trimers;
i. Load 900 grams of the filtered product into a 2,000 ml flask;
j. Product was heated to 200° C. and applied a vacuum of 10 mmHg;
k. 410 grams of isomerized fatty acids were distilled.

### Analysis of Isomerized and Distilled Fatty Acid:

| Acid value | 184 mgKOH/gram |
|---|---|
| Iodine index | 76 cgI₂/100 grams of product |
| Saponification index | 181.2 mgKOH/gram |
| Gardner Color | 3.0 |

### Isomerization of Soy Fatty Acids:

a. In a 2-liter autoclave, 1000 grams of soy fatty acid (SOFA) with an iodine value of 126 cgI₂/100 grams of product and an acidity value of 193 mgKOH/gram were loaded;
b. Added 80 grams of montmorillonite B clay and 5 grams of lithium hydroxide;
c. Pressurized with 5 kg of nitrogen;
d. Reactor temperature was gradually increased until reaching 270° C.;
e. During the heating process, the reactor pressure was relieved to maintain the pressure at 5 bar;
f. Product was reacted for 3 hours at 760° C.;
g. After this period it was cooled to 90° C. and filtered;
h. Product composition 41.1% monomers, 51.8% dimers, 7.1% trimers;
i. Load 900 grams of the filtered product into a 2,000 ml flask;
j. Product was heated to 200° C. and applied a vacuum of 10 mmHg;
k. 508 grams of isomerized fatty acids were distilled;

### Analysis of Isomerized and Distilled Fatty Acid:

| Acid value | 186 mgKOH/gram |
|---|---|
| Iodine index | 78 cg I2/100 grams of product |
| Saponification index | 186.2 mgKOH/gram |
| Gardner Color | 4.0 |

### Isomerization of Soy Methylester:

a. In a 2-liter autoclave, 1000 grams of soy methyl ester (FAME) were loaded with an iodine index of 126 cg I₂/100 grams of product and a saponification index of 192 mgKOH/grams of product;
b. Added 60 grams of montmorillonite B clay and 5 grams of lithium hydroxide;
c. Pressurized with 5 kg of nitrogen;
d. Reactor temperature was gradually increased until reaching 240° C.;
e. During the heating process, the reactor pressure was relieved to maintain the pressure at 5 bar;
f. Product was reacted for 5 hours at 240° C.;
g. After this period it was cooled to 90° C. and filtered;
h. Product composition 53.2% monomers, 40.2% dimers, 6.6% trimers;
i. Load 900 grams of the filtered product into a 2,000 ml flask;
j. Product was heated to 200° C. and applied a vacuum of 10 mmHg;
k. 520 grams of dimerized methyl esters were distilled.

### Analysis of Dimerized Methylester:

| Acidity value | 8.2 mgKOH/gram |
|---|---|
| Iodine index | 87 cg I₂/100 grams of product |
| Saponification index | 18.62 mgKOH/gram |
| Gardner Color | 3.0 |

### Isomerization of Soy Methylester (Fame):

a. In a 2-liter autoclave, 1000 grams of soy methyl ester (FAME) were loaded;
b. Added 25 grams of Beta zeolite calcined at 450° C.;
c. Pressurized with 8 bar of nitrogen;
d. Reactor temperature was gradually increased until reaching 280° C.;
e. During the heating process, the reactor pressure was relieved to maintain the pressure at 8 bar;
f. Product was reacted for 6 hours at 280° C.;
g. After this period it was cooled to 90° C.;
h. Tonsil filtration aid were added and the product was filtered;
i. Product composition: 78.3% isomerized methyl esters and 22.7% linear methyl esters

### Analysis of Isomerized Methylester:

| Acid value | 10.2 mgKOH/gram |
|---|---|
| Iodine index | 54.2 cg I₂/100 grams of product |
| Saponification index | 186.2 mgKOH/gram |
| Gardner Color | 3.0 |

### Isomerization of Talloil Fatty Acid (Tofa):

a. In a 2-liter autoclave, 1000 grams of talloil fatty acid (TOFA) were charged;
b. Add 30 grams of Beta zeolite calcined at 450° C.;
c. Pressurized with 8 bar of nitrogen;
d. Reactor temperature was gradually increased until reaching 270° C.;
e. During the heating process, the reactor pressure was relieved to maintain the pressure at 8 bar;
f. Product was reacted for 6 hours at 280° C.;
g. After this period it was cooled to 90° C.;
h. Tonsil filtration aid were added and the product was filtered;
i. Product composition: 84.3% isomerized fatty acids and 15.2% isomerized fatty acids.

### Analysis of Isomerized Fatty Acid:

| Acid value | 179.4 mgKOH/gram |
|---|---|
| Iodine index | 74.2 cg I₂/100 grams of product |
| Saponification index | 188.2 mgKOH/gram |
| Gardner Color | 3.0 |

j. 500 grams of the isomerized material are placed in a crystallizer and cooled to 2° C. forming a viscous and pasty mass;
k. Material after cooling is filtered at a temperature between 5° C. and 10° C. under vacuum;
l. 400 grams of clear liquid material were obtained;
m. Isomerized fatty acid content was 96.3%.
   Production of Esters and Polyesters from Isomerized Materials

### Synthesis of Ethyl Isooleate

400 grams of isomerized fatty acid with an active ingredient of 96.3% were esterified and ethyl oleate was produced with the following characteristics:

| Acid value | 0.36 mgKOH/gram |
|---|---|
| Saponification index | 189.4 mgKOH/gram |
| Iodine index | 3.2 cg I₂/100 grams of product |
| Solidification point | −14.8 Celsius |
| Gardner Color | 2 |

### Synthesis of Glyceryl Triisooleate

400 grams of the methyl ester from example 4 were transesterified and glyceryl triisooleate was produced with the following characteristics:

| Acid value | 0.10 mgKOH/gram |
|---|---|
| Saponification index | 189.4 mgKOH/gram |
| Iodine index | 8.6 cg I₂/100 grams of product |
| Solidification point | −14.8 Celsius |
| Gardner Color | 1 |

### Synthesis of Pentaerythritol Pentaisooleate:

400 grams of distilled isomerized fatty acid from example 1 were esterified with pentaerythritol with the following characteristics:

| Acid value | 0.08 mgKOH/gram |
|---|---|
| Saponification index | 179.4 mgKOH/gram |
| Iodine index | 8.6 cg I₂/100 grams of product |
| Solidification point | −8.8 Celsius |
| Gardner Color | 1 |

1. Process for the production of isomerized fatty acids and esters characterized in that it is from fatty chains obtained from triglycerides from soybean, corn, palm, canola, sunflower, peanut and rapeseed.

2. Process, according to claim 1, characterized in that the isomerization uses as catalysts earths of the montmorillonite/bentonite type or modified zeolites of the Beta type, calcined at 450° C. to obtain high reactivity and low porosity.

3. Process, according to claim 1, characterized in that montmorillonites or bentonites are used between 5% and 10% by weight, at temperatures of 240° C. to 270° C.

4. Process, according to claim 3, characterized in that said lands produce dimers and trimers in the range of 45% to 55% as co-products, which requires an additional distillation process to separate the isomerized materials.

5. Process, according to claim 4, characterized by the fact that the dimers and trimers are used to produce polyamides, polymers used as adhesives and paints.

6. Process, according to claim 2, characterized by in that, when using modified zeolites of the Beta type, they are used in the range of 1% to 5% with temperatures in the range of 250° C. to 290° C.

7. Process, according to claim 6, characterized in that there is no formation of dimers or trimers and the yield of isomerized materials is above 80%.

8. Process, according to claim 1, characterized in that the removal of linear materials to increase the content of isomerized materials is done through crystallization, and the content of isomerized materials is increased to above 94%.

9. Process according to claim 1, characterized in that the ethyl esters produced have a very low cloud point and are excellent lubricants, with spreadability and chemical stability characteristics suitable for use in cosmetic formulations replacing silicones and can be used as additives to reduce the cloud point of biodiesel made from triglycerides with high saturated material content.

10. Process, according to claim 1, characterized in that glycerin triesters produced from these materials are excellent humectants and can be used in cosmetic formulations such as skin creams as a hydrating agent, emollient and donor agent. of oil or components for oil-replenishing shampoos.

11. Process, according to claim 1, characterized in that pentaesters of the pentaisooleates/pentaisostearates type are excellent emollients, solvents with great wetting power and solvency that can be used in cosmetic formulations such as sunscreens, components for makeup and lipsticks, shampoos.
